(19) United States
(12) Patent Application Publication
(10) Pub. No.: US 2019/0309255 A1
(43) Pub. Date: Oct. 10, 2019

(54) SENSOR RECEPTACLE FOR A BIOREACTOR, AND BIOREACTOR WITH SENSOR RECEPTACLE, AND METHOD FOR PROPAGATION OR CULTIVATION OF BIOLOGICAL MATERIAL

(71) Applicant: SCHOTT AG, Mainz (DE)

(72) Inventors: Christian Ott, Ampfing (DE); Robert Hettler, Kumhausen (DE)

(73) Assignee: SCHOTT AG, Mainz (DE)

(21) Appl. No.: 16/379,133

(22) Filed: Apr. 9, 2019

(30) Foreign Application Priority Data
Apr. 9, 2018 (DE) 10 2018 108 325.4

(51) Int. Cl. C12M 1/00; C12M 1/34; C12M 1/12; G01N 21/65; G01N 21/21

(52) U.S. Cl. CPC C12M 43/00; C12M 41/00; C12M 37/00; G01N 21/21; C12M 23/38; G01N 21/65; C12M 23/22

(57) ABSTRACT

A sensor receptacle for a bioreactor and to a bioreactor having a sensor receptacle are provided, as are methods of use. The sensor receptacle has a window and extends at least partially in a through-opening of a container for holding fluid media containing biological material. The sensor receptacle and the window seal the through-opening.

### CROSS REFERENCE TO RELATED APPLICATIONS

This application claims benefit under 35 USC § 119 of German Application 10 2018 108 325.4, filed Apr. 9, 2018, the entire content of which are incorporated herein by reference.

The present application incorporates by reference the entire content of US Application No. [Attorney Reference No. 2133.390USU] filed simultaneously with the present application and the entire content of German Application 10 2018 323.8 filed Apr. 9, 2018 on which it is based.

The present application incorporates by reference the entire content of US Application No. [Attorney Reference No. 2133.392USU] filed simultaneously with the present application and the entire content of German Application 10 2018 108 327.0 filed Apr. 9, 2018 on which it is based.

### BACKGROUND

### 1. Field of the Invention

The invention relates to a sensor receptacle for a bioreactor and to a bioreactor comprising a sensor receptacle as well as to methods for propagation or cultivation of biological material.

### 2. Description of Related Art

Methods for producing biological material, such as for example biotechnological production processes involving the cultivation of microorganisms, animal and plant cells, are of increasing importance. Biotechnological production processes include, for example, the cultivation of microorganisms, animal and plant cells.

Conventionally, measuring probes are installed prior to the sterilization of the bioreactor, both in case of one-way bioreactors which are also referred to as single-use reactors, and in case of bioreactors intended for repeated use, which are also referred to as multi-use reactors in the art. Such measuring probes, for example pH probes, are generally calibrated prior to the sterilization.

An exchange of measuring devices for acquiring various physical, chemical or biological parameters during the production process, in particular during the cultivation, is commonly associated with high production risks up to the complete failure of a batch. A fault in autoclaving or during cultivation therefore usually implies a loss of process control.

A determination of substrate and product concentrations is very time-consuming in the conventional way. This is caused by the extraction of samples which is usually associated with a risk of contamination, as well as by resource-intensive off-line analysis.

DE 10 2010 063 031 A1 discloses a potentiometric sensor and a method for starting up a potentiometric sensor. In order to particularly simplify the use of a container serving as a single-use fermenter or single-use bioreactor, the potentiometric probe disclosed therein can be installed in a wall of the container, via a port, already before sterilization, for example by irradiation with gamma radiation, and can remain therein for the duration of storage and use.

DE 10 2006 022 307 A1 describes a one-way bioreactor comprising a reversibly externally attachable sensor arrangement for measuring a physical variable of a contained medium, and a sensor adapter for receiving an electronic sensor arrangement that is interacting with the medium flowing through the peripheral line, via an inner interface of the sensor adapter, is integrated in at least one peripheral line of the bioreactor serving for the inlet and/or outflow of medium. Since the measurement can be made only in the peripheral line of the bioreactor, process control within the bioreactor is impossible with this arrangement.

DE 10 2010 037 923 A1 discloses a bioreactor arrangement for cells, comprising a closed bioreactor, a cell pellet carrier for receiving a cell pellet, and means for supplying nutrient solution into the cell pellet. This arrangement allows for contactless measurement of the oxygen content. For this purpose, oxygen probes are excited to phosphorescence by a laser. The emitted phosphorescence signal is captured by the detector and fed to evaluation electronics. For this purpose, the bioreactor has light-transmitting windows, and the laser and the detector are arranged outside the bioreactor. An exchange of sensors or measuring equipment is not described in this document.

DE 10 2011 101 108 A1 describes a transflection probe for performing a transflection measurement on a fluid located in a rigid container, comprising a probe shaft having a light guide path in the interior thereof, and having an open flow chamber on the front end face, with a reflective plate arranged opposite the front end face of the probe shaft. The probe shaft is in the form of a rigid chamber which is sealed at its front end face by a transparent window and which has a first coupling device at its rear end for rigidly coupling a sensor module to the probe shaft. However, this coupling device is firmly connected to the open flow chamber and in particular to the reflective plate arranged opposite the front end face of the probe shaft, so that an exchange of sensor modules on a probe or the coupling of a sensor module to different probes of the same or of a different container in an alternating manner is possible. An exchange of sensors for measuring physical, chemical, or biological parameters is not disclosed.

### SUMMARY

Especially for one-way or single-use applications, there is no view port available on the bioreactor which would meet the optical requirements for suitable measurement technology, in particular the requirements on transmittance and freedom from distortion, and would support the sensor or probe in an exchangeable manner and mechanically reliable at the same time.

There is at present no prior art system on the market which would satisfy the requirement to hermetically isolate, in a reliably manner, the sterile cultivation area from the measurement area while allowing the use of sensors for different measuring techniques.

The invention is based on the object to provide a sensor receptacle, also referred to as a sensor port, and a bioreactor equipped therewith, which allow to flexibly exchange different sensors without thereby opening an access to the interior of the bioreactor.

A bioreactor according to the invention comprises a container for holding fluid media containing biological material, a feedthrough with a through-opening between the interior of the container for holding fluid media containing biological material and the exterior of the container for holding fluid media containing biological material, and a sensor receptacle for supporting at least one sensor, which extends at least partially in the through-opening of the feedthrough.

Preferably, the feedthrough comprises a standard port, such as an Ingold port, a Broadly James port, a B. Braun safety port, or a port in compliance with a different standard. Such ports each have an opening of a defined diameter, which typically connects the interior of a bioreactor to the exterior thereof or opens to the exterior.

Generally, the container for holding fluid media containing biological material may be the container of a multi-use bioreactor intended for repeated use.

Advantageously in this case, the container for holding fluid media containing biological material comprises stainless steel or is made of stainless steel.

Alternatively, the container for holding fluid media containing biological material may also be the container of a single-use bioreactor intended for one-way use.

In this case it is advantageous if the container for holding fluid media containing biological material comprises a plastic, in particular a sterilizable plastic, or is made of a plastic, in particular a sterilizable plastic. This plastic may comprise a polymeric material and may in particular be made of a suitable material which withstands gamma sterilization or chemical sterilization with ETO.

Suitable plastics include polyester elastomers with EVOH (ethylene vinyl alcohol copolymer) or polyethylenes, for example. A mixture of materials can be used in this case, for example a layer system comprising an outer layer which provides mechanical stability. A gas-tight intermediate layer may then adjoin an inside biocompatible layer.

In order to meet the stringent requirements for the production of biopharmaceuticals, the material of the bioreactor and of the sensor receptacle may each be selected so as to comply with the following standards: FDA approved materials (ICH Q7A, CFR 211.65(a)—Code of Federal Regulations, USP Class, animal derivative free, bisphenol A free); EMA (European Medicines Agency) EU GMP Guide Part II approved materials; Sectoral chemical resistance—ASTM D 543-06; and Biocompatibility, e.g. referred to US Pharmacopeia or tests referred to ISO 10993.

In order to achieve favorable conditions for measurements and a mechanically stable support of the sensor receptacle and in particular of a sensory device supported in the sensor receptacle, the sensor receptacle may extend in the feedthrough, in particular in the through-opening thereof, in a form-fitting manner with respect to the through-opening of the feedthrough, while the sensor receptacle together with its window seals the through-opening.

The invention furthermore encompasses a sensor receptacle for supporting at least one sensor for a bioreactor, in particular for a bioreactor as described above, which sensor receptacle comprises a holder body with a sensor receiving area, on which a window is arranged which includes a transparent element that is transmissive to electromagnetic radiation. This makes it possible to capture optical signals or measured parameters using different sensory devices which are supported so that they can be exchanged even during cultivation and so that the risk of contamination is considerably reduced.

Particularly advantageously, the bioreactor is autoclavable together with the sensor receptacle for supporting at least one sensor, in particular autoclavable while the sensor receptacle is held at least partially in the through-opening of the feedthrough, because this permits to exclude with a very high degree of certainty a contamination with biologically active or interacting material.

For this purpose, the sensor receptacle for supporting at least one sensor is in particular adapted to be autoclavable as well.

Surprisingly, it has been found that 3,500 autoclaving cycles at 2 bar and 134° C. were possible with the sensor receptacles for supporting at least one sensor as disclosed herein.

In the context of the present disclosure, autoclavable is understood to mean autoclavable in the sense of DIN EN ISO 14937; EN ISO 17665, which applies to medical devices.

Advantageously, the holder body has a cylindrically symmetrical shape, in particular a columnar shape, and has a through-opening which is sealed fluid-tightly, in particular hermetically, by the window on the side associated with the interior of the bioreactor. Such hermeticity also helps to ensures a contamination-free operation of the respective bioreactor.

Advantageously, the transparent element of the window exhibits a transmittance of greater than 80%, most preferably greater than 90%, in a spectral range of wavelengths between 250 and 2000 nm.

In the preferred embodiments, the transparent element of the window may comprise glass or may be made of glass.

Then, the glass of the transparent element of the window comprises or is made of quartz glass or borosilicate glass.

If the transparent element of the window is secured to a main body by a glass-to-metal seal (GTMS) compression glass seal, and the main body is secured to the holder body of the sensor receptacle, a mechanically and thermally stable connection is provided, which even withstands autoclaving in a durable and safe manner, as well as cleaning and sterilization using procedures that are known as CIP (Clean-In-Place) and SIP (Sterilization-In-Place).

Particularly preferably, the window is connected to the holder body by welding, in particular laser welding, so as to provide hermetical sealing.

In a particularly preferred embodiment, the transparent element of the window has a sheet-like shape and in particular plane-parallel main surfaces.

In further preferred embodiments, the transparent element of the window is one of plano-convex, plano-concave, biconvex, biconcave, convexo-concave, or concavo-convex. Especially in the latter case, the transparent element of the window may form part of an imaging system associated with the window.

The holder body may have a radially extending lateral shoulder, in particular in order to provide a stop in the axial direction for a standard port on the bioreactor.

In this case, when the sensor receptacle is installed in the through-opening, the axial distance between the transparent element of the window and the inner surface of the bioreactor can be defined by the axial distance of the transparent element of the window to the lateral shoulder. In this case, when the holder body is installed in the through-opening, in particular when the lateral shoulder abuts on a standard port, the transparent element of the window is advantageously arranged within the bioreactor. A set of holder bodies with different axial distances between the transparent element of the window and the lateral shoulder can thereby allow to selectively axially position the transparent element of the window within the bioreactor. In the context of the present disclosure, axial distance is understood to mean a distance measured or indicated in the direction of the axis of symmetry of the cylindrically symmetrical holder body of the sensor receptacle.

Advantageously, the holder body may comprise a flange, in particular a standard flange, which preferably extends radially outwardly and defines a contact surface for a sealing means in the axial direction. By way of example, this contact surface may be a contact surface as used for example in an Ingold port, a Broadly James port, a B. Braun safety port, or a port in compliance with a different standard.

The sensor receptacle may have associated therewith a covering cap having a contact surface for a sealing means, which forms part of a sensor, for example.

Advantageously, the flange of the holder body and a flange of the covering cap each form a portion of an annular shoulder extending obliquely at least in sections thereof.

A sensor associated with sensor receptacle may have at least a first sensor portion which can be fitted in the through-opening of the holder body, preferably in a positively fitting manner, and which comprises at least one sensory device.

In preferred embodiments of a sensor for the sensor receptacle described herein, the sensory device comprises an image guide, in particular a fiber image guide.

However, the sensory device may as well comprise an imaging optical system, in particular in combination with an image capturing device. This may be, for example, an image capturing sensor of a digital recording device, which can be used for documentation or for real-time monitoring.

If the sensory device comprises a spectrometer, this permits to perform photoluminescence measurements, for example, permitting to separate the light of a photoemission exciting laser from the emitted photoluminescence light.

Anyhow, what is generally decisive for the product yield, besides such parameters as temperature which can also be detected spectrally, is the monitoring or metrological monitoring of substances that are relevant for metabolism and product generation, and this in real time.

A preferred method for propagation or cultivation of biological material comprises the introducing of fluid and in particular of biological material or of a precursor of biological material into a bioreactor, as described herein, and the capturing of a physical, chemical, or biological parameter using a sensor receptacle as described herein and a sensor as described herein.

A further preferred method for propagation or cultivation of biological material comprises the introducing of fluid media containing biological material or of a precursor of biological material into a bioreactor as disclosed above, in particular into a container of the bioreactor for holding fluid media containing biological material, wherein the container for holding fluid media containing biological material has a feedthrough with a through-opening between the interior of the container for holding fluid media containing biological material and the exterior of the container for holding fluid media containing biological material, and the method furthermore comprises, prior to the introducing of the fluid media containing biological material or of a precursor of biological material, the mounting of a sensor receptacle which comprises a window including a transparent element that is transparent to electromagnetic radiation, at or in the through-opening of the feedthrough of the container for holding fluid media containing biological material.

The methods for propagation or cultivation disclosed herein may very advantageously comprise the production of pharmaceuticals, in particular of biopharmaceuticals.

Preferably, the bioreactor is sterilized after the mounting of the sensor receptacle.

According to such method, the bioreactor is equipped with a sensor after the mounting of the sensor receptacle, and the sensor is introduced at least partially into the sensor receiving area of the holder body.

Sensory readings of a sensor that is disposed in a sensor receptacle as disclosed herein can then be captured, and sensors can be exchanged even during the cultivation or propagation, and it is in particular possible to use various types of sensors.

Examples of this include microscope probes, more generally sensors which are able to detect electromagnetic radiation, for example in the ultraviolet or in the ultraviolet and visible spectral range, and infrared sensors. Furthermore, the sensors may comprise devices for turbidity measurement and for Raman spectroscopy for measuring the energy shift. In case of the latter sensor, the sensor may also include an excitation light source for Raman spectroscopy.

Furthermore, the sensor may also comprise a sensor for measuring the polarization change of emission light emitted by the biological material.

Advantageously, the sensor receptacle may accommodate at least one sensor, for measuring the radiation intensity and/or the wavelength of the electromagnetic radiation in the interior of the bioreactor, in particular in the interior of the container for holding fluid media containing biological material, during an operating state.

If the sensor receptacle accommodates at least one sensor, and the radiation intensity and/or the wavelength of the electromagnetic radiation inside the bioreactor, in particular in the interior of the container for holding fluid media containing biological material, is measured in a spatially resolved manner in the operating state, this allows, for example, to capture and to specifically influence metabolic processes in a spatially resolved manner.

If electromagnetic radiation of a defined wavelength, preferably 250 nm, is irradiated into the bioreactor for a defined period of time, and a radiation intensity and/or a wavelength of the electromagnetic radiation is measured inside the bioreactor over a broad range of wavelengths or selectively at a particular wavelength, in particular at 270 nm, this allows to detect portions of the light emitted due to fluorescence and to evaluate them with regard to defined metabolic processes within the bioreactor.

Advantageously, the devices and methods described herein allow to exchange a sensor, for example during the cultivation of biological material in the container of the bioreactor, in particular without altering the sterility conditions within the container of the bioreactor during an exchange or replacement of the sensor, which sterility conditions are therefore maintained.

Advantageously, in a method of a preferred embodiment, the bioreactor can be autoclaved together with its container for holding fluid media containing biological material and together with the sensor receptacle for supporting at least one sensor, while the sensor receptacle for supporting at least one sensor is held at least partially in the through-opening of the feedthrough, because in this case contamination of the bioreactor can be avoided with much higher probability than hitherto, and the autoclaving can be performed with a single operation. Consequently, if the autoclaving is carried out shortly before the bioreactor is filled with the biological material, the risk of an interim introduction of contaminants is also reduced.

The publication “Optical Sensor Systems for Bioprocess Monitoring” by Stefan Marose, Carsten Lindemann, Roland Ulber, and Thomas Scheper, TIBTECH JANUARY 1999 (VOL 17), teaches that the measurement of optical density is the most universal instrument for in-situ mass control in a bioreactor. The present invention greatly mitigates drawbacks of such devices, in particular with regard to process reliability.

With the methods presently disclosed it is possible to use in particular photo- or mixotrophic microorganisms modified by mutagenesis, in particular also microalgae, yeasts, and bacteria.

### DESCRIPTION OF THE DRAWINGS

The invention will be described in more detail below by way of preferred embodiments and with reference to the accompanying drawings, wherein:

FIG. 1 is a cross-sectional view of a first preferred embodiment of a sensor receptacle for supporting at least one sensor, which sensor receptacle is arranged in a through-opening of a container for holding fluid media containing biological material, although the container is shown only partially, the view being taken along a sectional plane illustrated as sectional plane A-A in FIG. 4;

FIG. 2 is a view obliquely from above, but as seen from the interior of a container for holding fluid media, of the first preferred embodiment of the sensor receptacle for supporting at least one sensor, which sensor receptacle is arranged in a through-opening of the container for holding fluid media containing biological material, although the container is shown only partially;

FIG. 3 is a cross-sectional view of the first preferred embodiment of the sensor receptacle for supporting at least one sensor, which sensor receptacle is arranged in a through-opening of a container for holding fluid media containing biological material, although the container is shown only partially, the view being taken along a sectional plane illustrated as sectional plane B-B in FIG. 2;

FIG. 4 is a cross-sectional view of the first preferred embodiment of the sensor receptacle for supporting at least one sensor, which sensor receptacle is arranged in a through-opening of a container for holding fluid media containing biological material, although the container is shown only partially, the view being taken along a sectional plane illustrated as sectional plane C-C in FIG. 2;

FIG. 5 is a cross-sectional view of a second preferred embodiment of a sensor receptacle for supporting at least one sensor, which sensor receptacle is arranged in a through-opening of a container for holding fluid media containing biological material, although the container is shown only partially, the sectional plane being essentially as in FIG. 1 for the first embodiment;

FIG. 6 is a plan view of the second preferred embodiment of the sensor receptacle for supporting at least one sensor, but as seen from the interior of the container for holding fluid media, with the sensor receptacle arranged in a through-opening of a container for holding fluid media containing biological material, although the container is shown only partially;

FIG. 7 is a cross-sectional view of the second preferred embodiment of the sensor receptacle for supporting at least one sensor, which sensor receptacle is arranged in a through-opening of a container for holding fluid media containing biological material, although the container is shown only partially, the sectional plane extending vertically through the container for holding biological material, in front of the sensor receptacle and in front of the feedthrough;

FIG. 8 is a cross-sectional view of the second preferred embodiment of the sensor receptacle for supporting at least one sensor, which sensor receptacle is arranged in a through-opening of a container for holding fluid media containing biological material, although the container is shown only partially, the sectional plane extending horizontally through the container for holding biological material, in front of the sensor receptacle and in front of the feedthrough;

FIG. 9 is a perspective cross-sectional view taken along the sectional plane A-A of FIG. 4 of the first preferred embodiment of a sensor receptacle for supporting at least one sensor, which sensor receptacle is arranged in a through-opening of a container for holding fluid media containing biological material, although the container is shown only partially;

FIG. 10 is a perspective cross-sectional view taken along the sectional plane D-D of FIG. 5 of the second preferred embodiment of a sensor receptacle for supporting at least one sensor, which sensor receptacle is arranged in a through-opening of a container for holding fluid media containing biological material, although the container is shown only partially;

FIG. 11 is a detail view of an upper portion of FIG. 9;

FIG. 12 is a side view of a bioreactor with a sensor receptacle for supporting at least one sensor, which shows the container for holding fluid media containing biological material partially broken away;

FIG. 13 is a cross-sectional view of a further preferred embodiment of a sensor receptacle for supporting at least one sensor, which sensor receptacle is arranged in a through-opening of a container for holding fluid media containing biological material, although the container is shown only partially, the view being taken along the sectional plane illustrated as sectional plane E-E in FIG. 17, and the bioreactor being a multi-use bioreactor, wherein the sensor receptacle for supporting at least one sensor has a measuring chamber, in particular for turbidity measurements;

FIG. 14 is a view obliquely from above, but as seen from the interior of a container for holding fluid media, of the further preferred embodiment of the sensor receptacle for supporting at least one sensor, which sensor receptacle is arranged in a through-opening of the container for holding fluid media containing biological material, although the container is shown only partially;

FIG. 15 is a cross-sectional view of the further preferred embodiment of the sensor receptacle for supporting at least one sensor, which sensor receptacle is arranged in a through-opening of a container for holding fluid media containing biological material, although the container is shown only partially, the view being taken along a sectional plane illustrated as sectional plane F-F in FIG. 14;

FIG. 16 is a further view obliquely from below, but as seen from the interior of a container for holding fluid media, of the further preferred embodiment of the sensor receptacle for supporting at least one sensor, which sensor receptacle is arranged in a through-opening of the container for holding fluid media containing biological material, although the container is shown only partially;

FIG. 17 is a cross-sectional view of the further preferred embodiment of the sensor receptacle for supporting at least one sensor, which sensor receptacle is arranged in a through-opening of a container for holding fluid media containing biological material, although the container is shown only partially, the view being taken along a sectional plane illustrated as sectional plane G-G in FIG. 14;

FIG. 18 is a further perspective view obliquely from above, but as seen from the exterior of a container for holding fluid media, of the further preferred embodiment of the sensor receptacle for supporting at least one sensor, which sensor receptacle is arranged in a through-opening of the container for holding fluid media containing biological material, although the container is shown only partially;

FIG. 19 is a cross-sectional view of yet a further preferred embodiment of a sensor receptacle for supporting at least one sensor, which sensor receptacle is arranged in a through-opening of a container for holding fluid media containing biological material, although the container is shown only partially, with the sectional plane extending essentially as in FIG. 5, and the bioreactor being a single-use bioreactor, wherein the sensor receptacle for supporting at least one sensor has a measuring chamber, in particular for turbidity measurements;

FIG. 20 is a plan view of the yet further preferred embodiment of the sensor receptacle for supporting at least one sensor, which sensor receptacle is arranged in a through-opening of a container for holding fluid media containing biological material, although the container is shown only partially, the view being as seen from the interior of the container for holding fluid media;

FIG. 21 is a cross-sectional view of the yet further preferred embodiment of the sensor receptacle for supporting at least one sensor, which sensor receptacle is arranged in a through-opening of a container for holding fluid media containing biological material, although the container is shown only partially, with the sectional plane extending as in FIG. 7;

FIG. 22 is a further perspective view obliquely from above, but as seen from the interior of a container for holding fluid media, of the yet further preferred embodiment of the sensor receptacle for supporting at least one sensor, which sensor receptacle is arranged in a through-opening of the container for holding fluid media containing biological material, although the container is shown only partially;

FIG. 23 is a cross-sectional view of the yet further preferred embodiment of the sensor receptacle for supporting at least one sensor, which sensor receptacle is arranged in a through-opening of a container for holding fluid media containing biological material, although the container is shown only partially, with the sectional plane extending as in FIG. 10;

FIG. 24 is a further perspective view obliquely from below, but as seen from the exterior of a container for holding fluid media, of the yet further preferred embodiment of the sensor receptacle for supporting at least one sensor, which sensor receptacle is arranged in a through-opening of the container for holding fluid media containing biological material, although the container is shown only partially;

FIG. 25 is a schematic sectional view along the axis of symmetry S of a window described herein, for explaining the method of producing such a window;

FIG. 26 is a schematic sectional view taken along the axis of symmetry S of a sensor receptacle for supporting at least one sensor, showing part of the holder body of the sensor receptacle and a plane-parallel transparent element;

FIG. 27 is a schematic sectional view taken along the axis of symmetry S of a window of the sensor receptacle, showing part of the holder body of the sensor receptacle and a biconvex transparent element;

FIG. 28 is a schematic sectional view taken along the axis of symmetry S of a window of the sensor receptacle, showing part of the holder body of the sensor receptacle and a biconcave transparent element;

FIG. 29 is a schematic sectional view taken along the axis of symmetry S of a window of the sensor receptacle, showing part of the holder body of the sensor receptacle and a plano-convex transparent element;

FIG. 30 is a schematic sectional view taken along the axis of symmetry S of a window of the sensor receptacle, showing part of the holder body of the sensor receptacle and a plano-concave transparent element;

FIG. 31 is a schematic sectional view taken along the axis of symmetry S of a window of the sensor receptacle, showing part of the holder body of the sensor receptacle and a convexo-concave transparent element;

FIG. 32 is a schematic sectional view taken along the axis of symmetry S of a window of the sensor receptacle, showing part of the holder body of the sensor receptacle and a concavo-convex transparent element;

FIG. 33 is a detail view similar to that of FIG. 9, but illustrating a further embodiment in which a window forms part of a microscopic device associated therewith, in particular of a microscope probe;

FIG. 34 is a schematic sectional view along the axis of symmetry S of a window described herein, with the transparent element thereof comprising quartz glass, for explaining the method of producing such a window;

FIG. 35 is a top plan view of the window shown in FIG. 34, in which the transparent element comprises quartz glass; and

FIG. 36 shows a detail of FIG. 23 corresponding to the area within the rectangle K of FIG. 23.

### DETAILED DESCRIPTION

In the following detailed description of preferred embodiments, the same or equivalent components are designated with the same reference numerals in the figures in each case.

However, the figures are not drawn to scale, for the sake of clarity.

For the sake of brevity, the container for holding fluid media containing biological material will also be referred to as a container for holding fluid media hereinafter, or else, even more briefly, only as a container.

Insofar as the term ‘fluid media’ is used in the context of the present disclosure, this takes account of the fact that more than one fluid constituent may be provided in a bioreactor, for example a carrier fluid which may contain the biological material or a precursor of the biological material, but which may also include fluid components of the biological material itself or further nutrient solution. However, if there is only one carrier fluid and no other fluid component contained in the bioreactor, the term ‘fluid media’ is meant to encompass this carrier fluid also in the singular, without the need of additionally having further fluid constituents.

In the context of the present disclosure, the biological material generally comprises prokaryotic and eukaryotic cells, such as mammalian cells, photo-, hetero-, and mixotrophic microorganisms, and is for instance provided in the form of microalgae, for example blue-green algae or cyanobacteria, and in particular also comprises photo- or mixotrophic microorganisms that have been modified by mutagenesis.

Referring first to FIG. 12 which shows a multi-use bioreactor designated by reference numeral 1 as a whole, on which a sensor receptacle is disposed, designated by reference numeral 2 as a whole, for supporting at least one sensor.

The bioreactor 1 comprises a container 3 for holding fluid media that contains biological material, and both the fluid medium 4 or fluid media 4 and the punctiform biological material 5 can be seen within the circle K delimiting a broken cross-sectional view of the container 3.

Generally, the container 3 may be the container of a multi-use bioreactor 1 intended for repeated use, as shown in FIG. 12 by way of example, or else of a single-use bioreactor intended for one-way use.

In the case of a multi-use bioreactor, the container 3 advantageously comprises stainless steel or is made of stainless steel. The sensor receptacle 2, which will be described in more detail below, may also comprise stainless steel, or at least one or more of its components and for example the main body 10 of the windows 11 disclosed herein and shown in FIG. 7 may comprise or be made of stainless steel.

Any stainless steel may be used for this purpose, especially also austenitic and ferritic stainless steels, but preferably only as far as they remain rust-free when practicing the invention.

The stainless steel may preferably also comprise or may entirely consist of 316L pharmaceutical grade steel.

Furthermore, titanium, tantalum, nickel, and Monel alloy with a high copper content may also be used, in principle, and when the titanium is used for the illumination device, in particular for the housing body thereof, it may also be enameled.

In the case of single-use reactors 1, the container 3 for holding fluid media containing biological material may comprise a plastic, in particular a sterilizable plastic, or may be made of a plastic, in particular of a sterilizable plastic. In this case, the container 3 does not have the shape shown in FIG. 12 but may even be in the form of a bag.

Furthermore, more than one port 6 may be provided on the container 3, which is also referred to as a feedthrough 6 in each case.

Preferably, these feedthroughs 6 comprise a standard port, as is the case in particular also in the embodiment shown in FIG. 12, for example an Ingold port, a Broadly James port, a B. Braun safety port, or a port in compliance with a different standard.

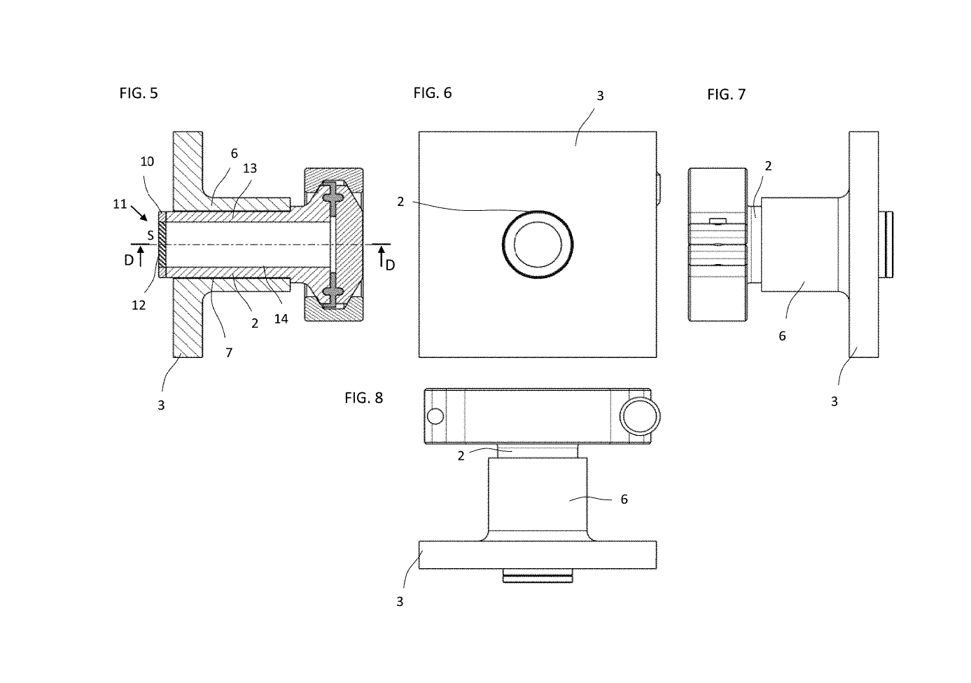

In the case of one-way reactors or single-use reactors, as shown in FIGS. 5 to 8, 10, and 19 to 24, for example, the feedthrough 6 may also comprise a tri-clamp port, sanitary clamp port, or a manufacturer-specifically adapted port.

The feedthroughs 6 each have a through-opening 7 of a defined diameter, which typically connects the interior of a bioreactor 1 to the exterior thereof or provides access to the container 3.

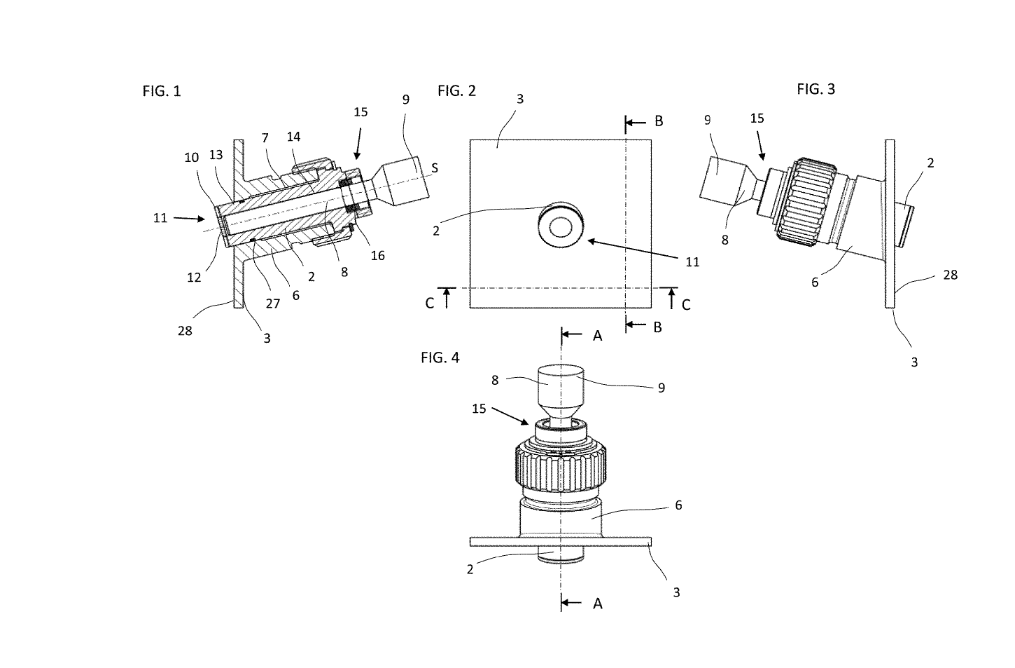

These through-openings 7 of ports 6 can be seen particularly well in FIGS. 1, 5, 9, 10, and 11, for example.

A sensor receptacle 2 for supporting at least one sensor 8, for example an image capturing device or a microscope probe 9, is arranged in at least one through-opening 7, and the sensor receptacle 2 is held at least partially extending in the through-opening 7 of the port 6 or feedthrough 6, as portions of the sensor receptacle 2 may also protrude into the interior of the container 3, and portions may also protrude beyond the through-opening 7 to the outside of the container 3.

The embodiment of the invention thus comprises a bioreactor 1 with a container 3 for holding fluid media 4 containing biological material 5, a feedthrough 6 or port 6 with a through-opening 7 between the interior of the container 3 for holding fluid media 4 containing biological material 5 and the exterior of the container 3 for holding fluid media 4 containing biological material 5, and with a sensor receptacle 2 for supporting at least one sensor 8, which extends at least partially in the through-opening 7 of the feedthrough 6 or port 6.

The sensor receptacle 2 comprises a holder body 13 with a sensor receiving area 14, in particular in the form of a through-opening, where a window 11 is arranged which includes a transparent element 12 that is transmissive to electromagnetic radiation, see also FIGS. 1 and 5, for example.

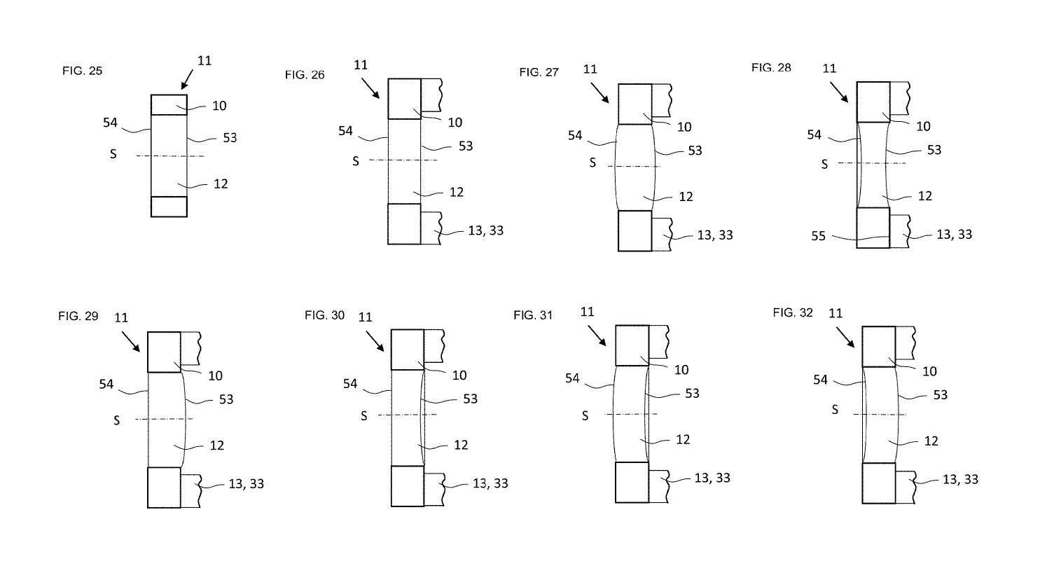

The window 11 comprises a main body 10 which comprises steel or is made of steel and holds the transparent element 12, as will be described in more detail below with reference to FIGS. 25 through 32.

The bioreactor 1 may also have a plurality of feedthroughs 6, with a respective sensor receptacle 2 arranged in each of those feedthroughs.

In this case, the same type of sensor 8 may be arranged in the sensor receptacle 2, or else different embodiments of sensors 8 may be arranged therein.

For example, a first preferred embodiment comprises a microscope probe 8, 9 known per se to a person skilled in the art, which may include an image capturing device and which has a substantially cylindrical shape and is supported in the sensor receptacle 2 in a detachable manner, but nevertheless firmly.

The sensor receptacle 2 for supporting at least one sensor 8 will now be described in more detail with reference to FIGS. 1 to 4.

As can be seen particularly well from FIG. 1 and FIG. 5, the sensor receptacle 2 comprises a holder body 13 which has a cylindrically symmetrical shape, in particular a columnar shape, and is preferably formed in one piece.

The holder body 13 has a preferably cylindrical sensor receiving area 14 which is sealed fluid-tightly, in particular hermetically, by a window 11 on the side associated with the interior of the bioreactor 1.

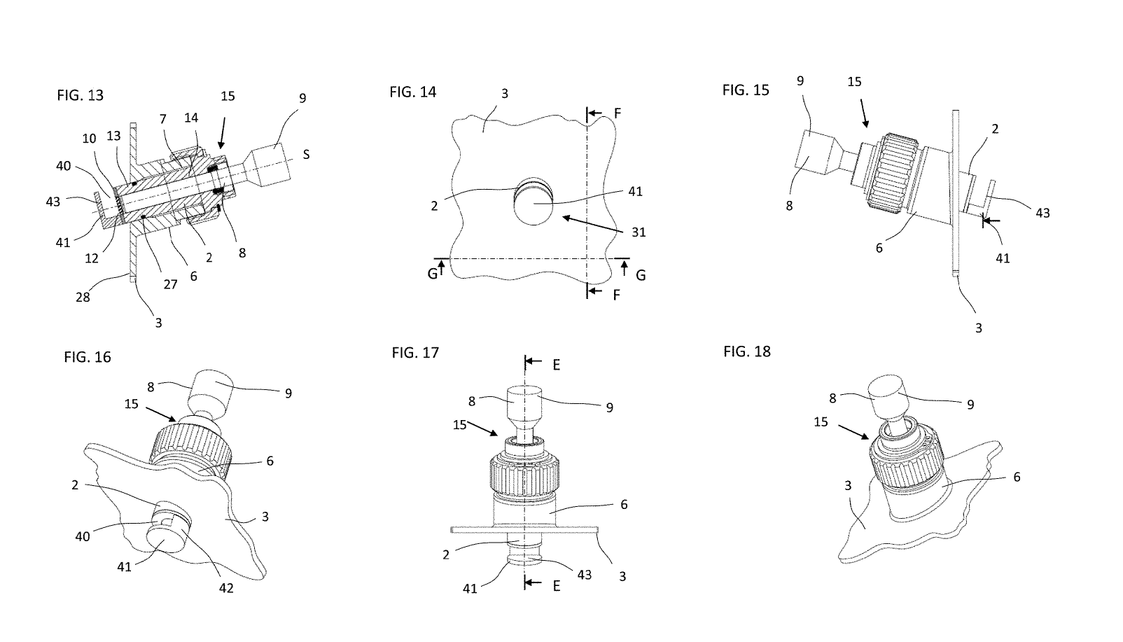

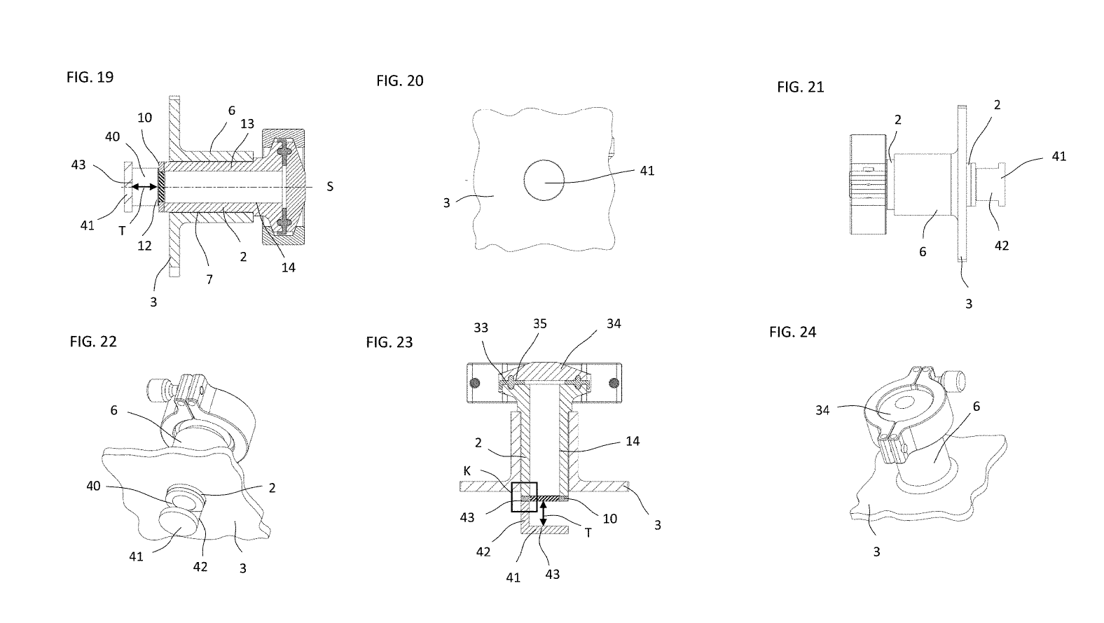

FIGS. 13 to 18 show a further embodiment, of a multi-use bioreactor 1 for repeated use which comprises a sensor receptacle 2 for supporting at least one sensor, and FIGS. 19 to 24 show yet a further embodiment, of a single-use bioreactor 1 for one-way use which comprises a sensor receptacle 2.

Both embodiments comprise a respective measuring chamber 40, in particular for turbidity measurements, see FIGS. 19 and 20, for example.

Measuring chamber 40 is defined by a foot portion 41 of substantially L-shaped cross section in combination with the window 11, in particular with the main body 10 and the transparent element 12 thereof.

Foot portion 41 comprises a base portion 42 which is secured to the holder body 13 preferably in a releasable manner and extends in the direction of the axis of symmetry S. At the distal end of the base portion 42, that is the most distant end from the holder body 13, the foot portion 41 defines a plane end face 43 extending perpendicular to the axis of symmetry S and delimiting the measuring chamber 40 in the direction of the axis of symmetry.

The end face 43 and the transparent element 12 are arranged with a distance T from each other, so that when using parallel light which propagates in the direction of the axis of symmetry S, a volume is defined by the surface of the transparent element 12 and the distance T, which volume is located within the container 3 for holding fluid media and can be used for turbidity measurements.

However, if no parallel light is used for the measurement but divergent light, a measurement volume is nevertheless defined by the area of surface 43 and the distance T, which can be used for turbidity measurements in this embodiment as well.

However, due to the static geometry, a non-changing measurement volume can generally always be calibrated first, for example only with a nutrient solution without the biological material, and can then be used for respective turbidity measurements after the biological material 5 has been introduced into the bioreactor 1.

The foot portion 42 may be permanently secured to the main body 10 of the window 11, for example by a welded connection, see for example FIG. 23.

Alternatively, the foot portion 41 may also be provided in a detachably secured manner, as shown in FIG. 36 by way of example. For this purpose, foot portion 41 may have a bore 44 of defined fit in its base portion 42, into which a dowel pin 45 extends, which is firmly held in the holder body 13 and is preferably also firmly held in the main body 10 of the window 11. As can be clearly seen in FIG. 5, for example, window 11 comprises a transparent element 12 which is secured in a main body 10 in a fluid-tight, in particular hermetically sealed manner.

In the sense of the present disclosure, an item or else a connection between two items, for example between the transparent element 12 and the main body 10 of window 11, shall be considered as hermetically sealed or fluid-tight if it exhibits a leak rate of less than 1*10^{—3} mbar·l/sec at room temperature when exposed to He on one side and to a pressure difference of 1 bar.

The transparent element 12 is transparent to electromagnetic radiation, since it comprises glass or is made of glass, and the glass preferably comprises or is made of quartz glass or borosilicate glass.

The holder body 13 together with the window 11 defines a holder 15 for the sensor 8, in which the sensor 8 is held in a detachably mounted manner.

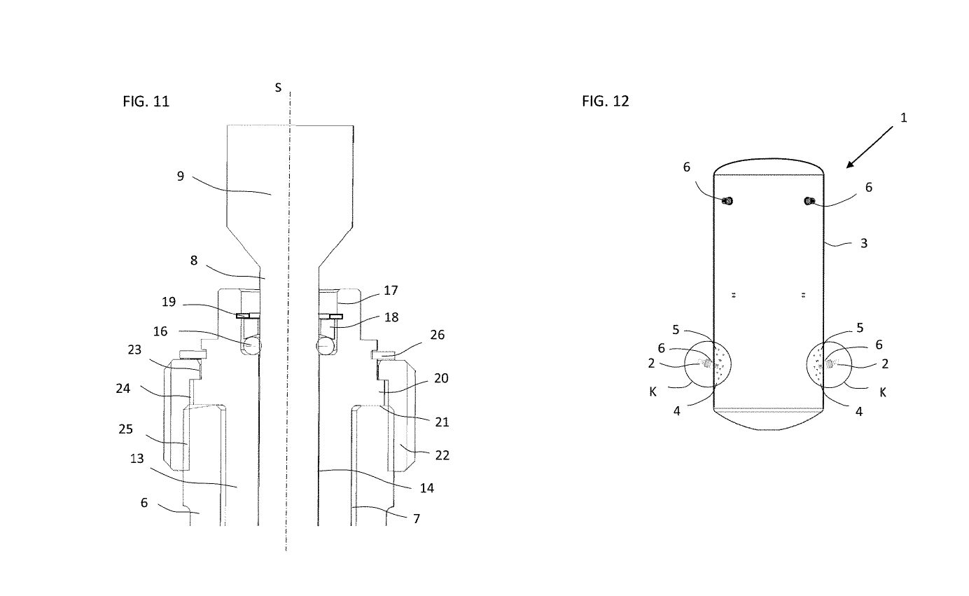

For this purpose, the holder body 13 may establish a frictional connection to the sensor 8, preferably by means of a frictional element 16, preferably an O-ring, as can be seen in FIG. 1 and FIG. 11, for example.

Frictional element 16 is held in a cylindrical recess 17 of the holder body 13 by a substantially annular pressure element 18 which exerts a defined adjustable force to the frictional element in the axial direction of the holder body 13.

With the resulting defined deformation of the frictional element 16, the sensor 8 can be held within the sensor receiving area 14 with a defined resulting force which reliably ensures the position of the sensor while still allowing for rapid manual removal thereof from the sensor receiving area 14 or rapid manual introduction into the sensor receiving area 14.

The annular pressure element 18 is fixed in its position in a defined manner by a snap ring 19. The holder body 13 itself has a radially extending lateral shoulder 20 abutting on an upper flange 21 of the feedthrough 6 in a form-fitting manner.

Thus, the sensor receptacle 2 extends within the feedthrough 6 or through-opening 7 of port 6 in a form-fitted manner with respect to the feedthrough or in a form-fitted manner with respect to the through-opening 7 of port 6.

The holder body 13 is releasably but stationary held on the port 6 by means of a cap nut 22 that has a cylindrical sensor receiving area 23 and engages over the radially extending shoulder 20.

For this purpose, the cap nut 22 has a thread 24 and the holder body 13 has a mating thread 25.

The cap nut 22 is captured on the holder body 13 by a snap ring 26 so as to be rotatable but only with little axial play, so it cannot be lost.

The sensor receptacle 2 can be quickly and safely mounted on the respective port 6 and also detached therefrom by rotating the cap nut 22.

By means of a sealing element 27, for example an O-ring, the holder body 13 is held in the through-opening 7 of the container 3 with a positive and frictional fit so as to seal the through-opening 7 fluid-tightly and preferably hermetically.

The axis of symmetry S of the holder body 13, which can be seen in FIGS. 1 and 5, for example, defines the axial or longitudinal direction here, which is referred to in the context of the present disclosure.

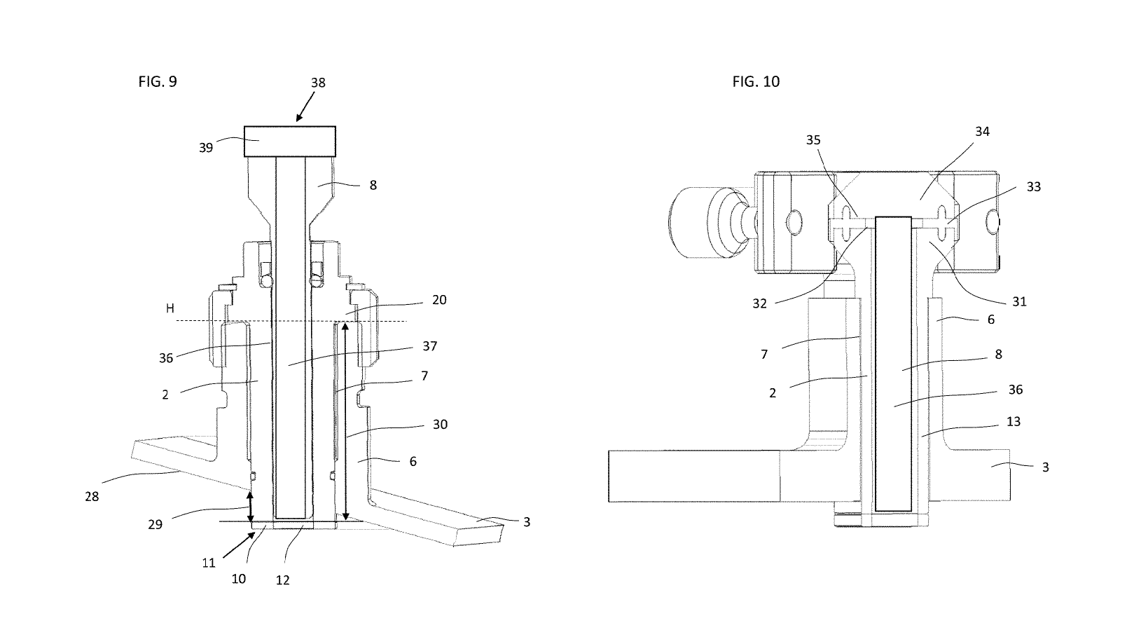

When a sensor receptacle 2 is placed in the through-opening 7 and preferably fixed therein as described above, see for example FIG. 9, the axial distance 29 of the window 11 and thus of the transparent element 12 to the inner surface 28 of the bioreactor 1 is defined by the axial distance 30 of the window 11 to the lateral shoulder 20.

The axial distance of the window 11 to the lateral shoulder 20 is measured starting from the underside of the radially extending lateral shoulder 20, as indicated by an auxiliary line H, to the upper surface of the main body 10 of window 11, as can be seen in FIG. 9, for example.

Here, the axial distance 29 of the window 11 to the inner surface 28 of the bioreactor 1 is the largest distance between the underside of main body 11 to the inner surface 28 of the bioreactor 1, i.e. to the inner surface 28 of the container 3 of the bioreactor 1.

When the holder body 13 is placed in the through-opening 7, in particular when the lateral shoulder 20 abuts against a standard port 6 as shown in FIGS. 1 and 9, for example, the transparent element 12 of the window 11 is preferably arranged within the bioreactor 1.

A set of holder bodies 13 with different axial distances 30 of the transparent element 12 of the window 11 to the lateral shoulder 20 can permit to place the transparent element 12 of the window within the bioreactor 1 at a selective axial position.

Thus, when using a plurality of sensor receptacles 2 each featuring a different axial distance 30 between the window 11 and the lateral shoulder 20, it is possible to capture different locations of the bioreactor 1. With this procedure and the use of multiple sensor receptacles 2, it is thus possible to capture a bioreactor 1 with its internal local processes in a considerably better way.

Reference is now made to FIGS. 5 and 10 which show a second preferred embodiment of a sensor receptacle 2, which is arranged on a single-use bioreactor 1 in which the container 3 of the bioreactor is made of plastics.

In this embodiment, the sensor receptacle 2, in particular the holder body 13 thereof, is welded to the container of the bioreactor 1, in particular welded to the through hole 7 of the bioreactor 1 without additives.

In order to increase the surface area and to improve the adhesive force, the holder body 13 may feature a knurling on its outer surface which contacts the plastic of the bioreactor 1.

As can be seen particularly well from FIG. 10, the holder body 13 has a flange 31, which in particular comprises a standard flange, which extends radially outwards and defines a contact surface 32 for a sealing means 33 in the axial direction.

Furthermore, in this embodiment, the sensor receptacle 2 comprises a covering cap 34 that has a contact surface 35 for a sealing means 33, which covering cap may be part of a sensor 8 and may preferably be capable of holding a sensor 8 in or on the sensor receptacle 2.

This covering cap 34 can be held on the flange 31 by conventional sealing and fastening means known for tri-clamp ports, with the sealing means 33 sandwiched therebetween, as will be known to a person skilled in the art. For example, sealing brackets that can be used for this purpose are standardized in compliance with ISO 2852 or DIN 32676.

This sensor 8 which is shown merely by way of example in FIGS. 9 and 10, may comprise a first sensor portion 36, for example, which can be fitted in the sensor receiving area 14 of the holder body 13, preferably in a positively fitting manner, and which comprises at least one sensory device.

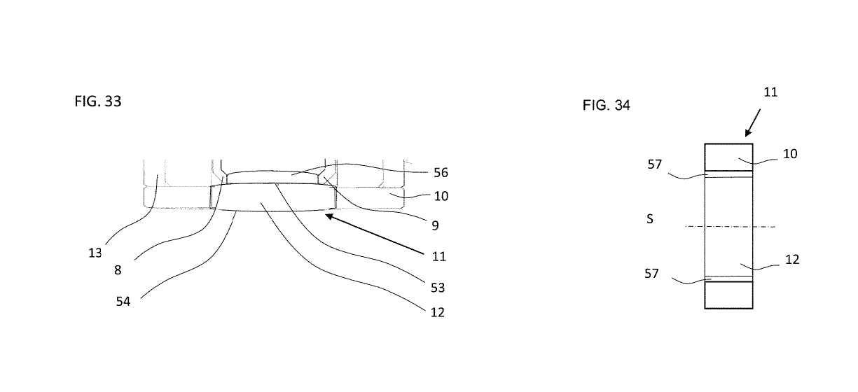

The sensory device may be a microscope probe 9, for example, which comprises an imaging optical system including an optical lens element 56 that is shown in FIG. 33, by way of example.

In a further embodiment, the microscope probe 9 may comprise an image capturing device, for example in the form of an image capturing photosensor.

Alternatively or additionally, the sensory device may comprise an image guide, in particular a fiber image guide 37, which is shown in FIG. 9 merely highly schematically and by way of example.

Alternatively, the sensory device may comprise a spectrometer 38, which is exemplified as a spectrometer head 39 flanged to the image guide 37 and optically coupled thereto.

However, if the sensor receptacle 2 has at least one sensor 8, in particular an image capturing device in a microscope probe 8 or associated with this microscope probe 8, as mentioned above, and if in the operating state this image capturing device is used to capture the radiation intensity and/or wavelength of the electromagnetic radiation in the interior of the bioreactor 1, in particular in the interior of the container 3, it is possible to measure the radiation intensity and/or the wavelength in a spatially resolved manner. Furthermore, for measuring the wavelength, coatings on the transparent element 12 will be described below, by way of example. Moreover, both optical and electronic type filters disposed within a microscope probe 8 can be used for this purpose in the image capturing device, for example.

Process control with the use of an in-situ microscope as a sensor 8 makes it possible here to acquire quantitative and morphological information about the respective cells of the biological material 5.

As a result, an optimization of the influencing parameters for an optimized or at least improved yield may directly be derived therefrom. Such parameters include, for example, the fumigation rate, the fumigation composition, temperature, pH, rX, concentrations of dissolved gases, mixing/stirrer speed, feed rate, feed composition, cultivation time, activating or inhibiting factors, and may also include further influencing factors.

In the present preferred embodiments, a window 11 or both windows 11 each provide a glass seal for a transparent element 12, preferably a Glass-To-Metal-Seal (GTMS) compression glass seal, as will be described below with reference to FIG. 25.

FIG. 25 shows a schematic sectional view along an axis of symmetry S of a window 11 described herein for explaining the method for producing such a window.

The window 11 shown in FIG. 25 comprises an annular or cylindrical body 10 made of steel, which encloses the transparent element 12 laterally while exerting thereon a compressive force which ensures a permanently hermetic connection between the transparent element 12 and the main body 10, which is sufficiently pressure-resistant and heat-resistant for the purposes of the present invention.

The transparent element of the window is secured to the main body 10 by the GTMS compression glass seal, and the main body is secured to the holder body 13 of the sensor receptacle.

Advantageously in this case, the window 11 is connected to the holder body 13 in a hermetically sealing manner, by welding, in particular laser welding.

For producing such a window, the transparent element comprising glass or made of glass is accordingly arranged within the main body 10, preferably in approximately its final shape, and is heated together with the main body until the glass of the transparent element 12 has exceeded its glass transition temperature T_{g} or hemisphere temperature and begins to fuse to the main body 10.

Once fused, the assembly of transparent element 12 and main body 10 is then cooled to room temperature, thereby forming a respective window 11 that includes a substantially sheet-like transparent element 12.

Since the stainless steel of the main body 10 has a thermal expansion coefficient that is greater than that of the glass of the transparent element 12, it will exert a compressive stress to the glass of the transparent element 12 as soon as the glass of the transparent element begins to solidify, which compressive stress is increasing with decreasing temperature.

Once the cooling has been completed, the main body 10 will then permanently and reliably hold the transparent element 12 in a hermetically sealed and temperature-stable manner, due to this quasi-frozen compressive stress.

Such a compression glass seal is also referred to as a glass-to-metal seal (GTMS) or GTMS compression glass seal in the present disclosure.

In such a glass-to-metal connection, the metal exerts pressure forces on the glass over the entire operating temperature range, in particular even at temperatures up to at least 121° C., preferably even up to 141° C., which pressure forces cause a compressive stress between the metal and the glass and help to ensure that the glass-to-metal connection remains permanently and reliably fluid-tight as well as hermetically sealed.

Furthermore, no gaps will arise with such glass-to-metal joints. By contrast, gaps may arise and may provide room for contamination that is often difficult to remove if conventional sealing means such as O-rings are used.

For this purpose, it is advantageous to provide for a difference in the coefficients of thermal expansion, which reliably ensures that the compressive stress between the glass of the transparent element 12 and the metal of the annular or cylindrical main body 10 of the window 11 is maintained at least over the range of operating temperatures.

This difference between the expansion coefficient CTE_{M} of the metal and the expansion coefficient CTE_{G} of the glass of the transparent element 12 may be less than 80*10⁻⁶/K, for example, preferably less than 30*10⁻⁶/K, or most preferably less than 20*10⁻⁶/K. Here, the coefficient of thermal expansion of the metal CTE_{M} should be greater than the coefficient of thermal expansion of the glass CTE_{G} in each case. In any cases, however, this difference should preferably be at least 1*10⁻⁶/K.

For example, quartz glass has a CTE_{G} of 0.6*10⁻⁶/K and can be combined, for example, with stainless steels that have a CTE_{M} of 17 to 18*10⁻⁶/K.

The bioreactor 1 and the sensor receptacle 2 are each autoclavable individually, or else the photobioreactor 1 and the sensor receptacle 2 are autoclavable together. This means that in particular the sensor receptacle including its windows 11 as disclosed herein is hermetically sealed so as to withstand a treatment with saturated steam at a temperature of 121° C., in particular also 141° C., so that ingress of saturated steam or fluids generated thereby into the device 2 is prevented.

In the context of the present disclosure, autoclavable is understood to mean autoclavable in the sense of DIN EN ISO 14937; EN ISO 17665, which applies to medical devices.

This also allows for the advantageous steaming-in-place (SIP) which will be known to a person skilled in the art.

In this permanent hermetical and heat-stable sealing state, the glass of the transparent element 12 can either be used directly, preferably after verifying the respective face or main surface 53, 54, or may be subjected to further surface processing procedures such as polishing or shaping grinding.

In this way, the respective transparent element 12 may have plane-parallel faces or main surfaces 53 and 54, or else the transparent element 12 may be shaped to become one of plano-convex, plano-concave, biconvex, biconcave, convexo-concave, or concavo-convex, as can be seen in the sectional views of FIGS. 26 to 32.

In the case of lower optical requirements on the surface quality, in particular for the beam paths used for the measurement with the embodiment described with reference to FIG. 10, the transparent element 12 may also be held in a corresponding negative mold which substantially already corresponds to the final shape thereof, during the fabrication process.

The main surfaces 53 and 54 may have a wavelength-selective coating which provides an optical bandpass or edge filter. This coating may be provided only on one or else on both main surfaces 53, 54.

Without such a coating, in particular without any coating, the transparent element 12 of at least one window 11 exhibits a transmittance of greater than 80%, most preferably greater than 90%, in a spectral range of wavelengths between 250 and 2000 nm.

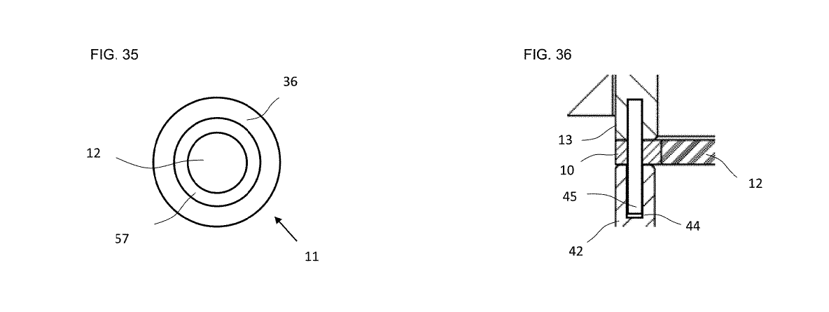

If the transparent element 12 of the window 11 consists of quartz glass or is made of quartz glass, as shown in FIGS. 34 and 35, for example, it is also possible, instead of a laser welding seam, to use a further glass 57 or glass solder 57, in particular lead-free glass 57 or glass solder 57 to hermetically seal the quartz glass to the annular or cylindrical main body 10 of the window 11, in particular fluid-tightly and hermetically.

The main body 10 of the window 11 is preferably connected directly to the holder body 13 in a hermetically sealed manner, by welding, in particular by laser welding, so that the holder body 13 is hermetically sealed at its lower end against the interior of the container 3.

The laser welding seam formed thereby is only indicated in FIG. 28 by way of example, by reference numeral 55.

Alternatively, the holder body 13 may also comprise or be made of a high temperature resistant plastic, in particular a thermoplastic material such as polyaryletherketone, in particular polyetheretherketone, PEEK.

If the holder body 13 comprises or is made of a high temperature resistant plastic, in particular a thermoplastic material such as polyaryletherketone, in particular polyetheretherketone, PEEK, this holder body 13 need not necessarily have to be completely hermetic as described in the context of the present disclosure, nevertheless it will be possible to achieve quite valuable operating and application times.

For example, the holder body 13 comprising PEEK or being made of PEEK may have a preferably columnar through-opening of circular cross-sectional shape, and a transparent element 12 which also has a circular outer lateral contour may have an outer diameter that is larger than the inner diameter of the circular through-opening of the holder body 13 by about 1/10. When the holder body 3 is heated to a temperature of about 200° C., the transparent element 12 can then be inserted into this through-opening, and when being cooled down, a compressive stress is resulting as described above, for example of about 38 MPa, which is still well below the yield strength of PEEK of 110 MPa.

A further preferred embodiment is resulting if the window shown in FIG. 27, for example, is used together with a microscope probe 8, as can be seen from FIG. 33, for example, and if this window 11 forms part of the microscopic device associated therewith, namely the microscope probe 8.

The transparent element 12 of the window 11 is part of an imaging system in particular associated therewith in this embodiment.

In this case, the lower main surface 54 alone or both main surfaces 54, 53 may have a beam- or wave-forming effect, for example, so that it is possible to achieve higher numerical apertures for the imaging beam path, in principle, since a larger effective angular range for the incoming electromagnetic radiation can be provided for the first optical lens element 56 of the microscope probe 8.

As a result, not only the resolution of the microscope probe 8 can be increased, but furthermore the total available intensity of electromagnetic radiation can be increased, so that when this window as shown in FIG. 27 is used in particular also for the embodiment shown in FIG. 10, an enhanced signal-to-noise ratio is resulting for the measurement results obtained thereby and is converted into electrical signals, for example.

The invention generally relates to a sensor receptacle for a bioreactor and to a bioreactor with a sensor receptacle, and to methods for propagation or cultivation of biological material using a sensor receptacle for supporting at least one sensor, which comprises a window and extends at least partially in a through-opening of a container for holding fluid media containing biological material, wherein the sensor receptacle with its window preferably seals the through-opening.

LIST OF REFERENCE NUMERALS:

| | |
|---|---|
| 1 | Bioreactor |
| 2 | Sensor receptacle |
| 3 | Container |
| 4 | Fluid medium or fluid media |
| 5 | Biological material |
| 6 | Port or feedthrough |
| 7 | Through-opening of container 3 |
| 8 | Sensor |
| 9 | Image capturing device |
| 10 | Main body |
| 11 | Window |
| 12 | Transparent element |
| 13 | Holder body |
| 14 | Sensor receiving area |
| 15 | Holder |
| 16 | Frictional element |
| 17 | Cylindrical recess |
| 18 | Annular pressure element |
| 19 | Snap ring |
| 20 | Radially extending lateral shoulder |
| 21 | Upper flange |
| 22 | Cap nut |
| 23 | Sensor receiving area |
| 24 | Thread |
| 25 | Mating thread |
| 26 | Snap ring |
| 27 | Sealing element |
| 28 | Inner surface |
| 29 | Axial distance |
| 30 | Axial distance |
| 31 | Flange |
| 32 | Contact surface |
| 33 | Sealing means |
| 34 | Covering cap |
| 35 | Contact surface |
| 36 | First sensor portion |
| 37 | Fiber image guide |
| 38 | Spectrometer |
| 39 | Spectrometer head |
| 40 | Measuring chamber |
| 41 | L-shaped foot portion |
| 42 | Base portion |
| 43 | Soldered or welded connection |
| 44 | Bore with defined fit |
| 45 | Dowel pin |
| 53 | Main surface |
| 54 | Main surface |
| 55 | Laser welding seam |
| 56 | Optical lens element |
| 57 | Glass or glass solder |
| S | Axis of symmetry |
| H | Auxiliary line |
| K | Rectangle defining a section |

What is claimed is:

1. A bioreactor, comprising:
a container having an interior configured to hold fluid media containing biological material and an exterior;
a feedthrough with a through-opening between the interior and the exterior;
a sensor receptacle that extends at least partially in through-opening, the sensor receptacle having a window that seals the through-opening.

2. The bioreactor of claim 1, wherein the container comprises a material selected from the group consisting of stainless steel, plastic, and a sterilizable plastic.

3. The bioreactor of claim 1, wherein the sensor receptacle extends in the feedthrough in a form-fitting manner with respect to the feedthrough.

4. The bioreactor of claim 1, wherein the bioreactor is autoclavable.

5. A sensor receptacle for supporting at least one sensor for a bioreactor, comprising:
a holder body with a sensor receiving area; and
a window arranged on the holder body, the window having a transparent element that is transmissive to electromagnetic radiation.

6. The sensor receptacle of claim 5, wherein the holder body has a cylindrically symmetrical shape and the sensor receiving area is hermetically sealed by the window.

7. The sensor receptacle of claim 5, wherein the transparent element exhibits a transmittance of greater than 80% in a spectral range of wavelengths between 250 and 2000 nm.

8. The sensor receptacle of claim 5, wherein the transparent element comprises a material selected from the group consisting of glass, quartz glass, and borosilicate glass.

9. The sensor receptacle of claim 5, wherein the transparent element is secured to a main body by a glass-to-metal seal (GTMS) compression glass seal, and the main body is secured to the holder body of the sensor receptacle.

10. The sensor receptacle of claim 5, wherein the window is laser welded to the holder body.

11. The sensor receptacle of claim 5, wherein the transparent element has a shape selected from the group consisting of plate-like with plane-parallel main surfaces, plano-convex, plano-concave, biconvex, biconcave, convexo-concave, and concavo-convex.

12. The sensor receptacle of claim 5, wherein the transparent element further comprises an imaging system.

13. The sensor receptacle of claim 5, wherein the holder body has a radially extending lateral shoulder, the lateral shoulder being positioned a first axial distance from the transparent element so that the first axial distance defines a second axial distance between the transparent element and an inner surface of the bioreactor.

14. The sensor receptacle of claim 5, wherein the holder body has a flange that extends radially outwardly and defines a contact surface for a seal in an axial direction.

15. The sensor receptacle of claim 5, further comprising a covering cap having a contact surface for a seal, the covering cap forming part of a sensor.

16. The sensor receptacle of claim 5, wherein the sensor receptacle is autoclavable.

17. A sensor for the sensor receptacle of claim 5, wherein the sensor has at least a first sensor portion that comprises a sensory device, the first sensor portion being sized to fit into the sensor receiving area of the holder body.

18. The sensor of claim 17, wherein the sensory device comprises a device selected from the group consisting of an image guide, a fiber image guide, an imaging optical system, an image capturing device, and a spectrometer.

19. A method for propagation or cultivation of biological material, comprising:
providing a bioreactor having an exterior and an interior, the interior being configured to hold a fluid media containing the biological material or a precursor of the biological material, the bioreactor having a feedthrough with a through-opening between the interior and the exterior;
mounting a sensor receptacle at least partially in the through-opening, the sensor receptacle having a holder body with a sensor receiving area and a window arranged on the holder body, the window having a transparent element that is transmissive to electromagnetic radiation, the window hermetically sealing the sensor receiving area;
placing a sensor in the sensor receptacle so that a first sensor portion of the sensor is fitted into the sensor receiving area of the holder body;
introducing the fluid media into the bioreactor; and
capturing a parameter of the fluid media and/or the interior of the bioreactor using the sensor, the parameter being selected from a group consisting of a physical parameter, a chemical parameter, a biological parameter, and any combinations thereof.

20. The method of claim 19, further comprising sterilizing the bioreactor after the step of mounting the sensor receptacle, but before the step of placing the sensor in the sensor receptacle.

21. The method of claim 19, wherein the step of capturing the parameter comprises measuring a radiation intensity and/or a wavelength of electromagnetic radiation in the interior of the bioreactor.

22. The method of claim 21, wherein the step of measuring the radiation intensity and/or the wavelength comprises measuring in a spatially resolved manner.

23. The method of claim 19, wherein the step of capturing the parameter comprises:
irradiating, into the bioreactor, electromagnetic radiation of a defined wavelength for a predefined period of time; and
measuring, after irradiating, a radiation intensity and/or a wavelength of the electromagnetic radiation in the interior of the bioreactor over a broad range of wavelengths or selectively at a particular wavelength.

24. The method of claim 19, further comprising:
removing, during the cultivation of biological material, the sensor from the sensor receptacle;
placing a second sensor in the sensor receptacle so that a first sensor portion of the second sensor is fitted into the sensor receiving area of the holder body; and
capturing a second parameter of the fluid media and/or the interior of the bioreactor using the second sensor.
